(19) United States
(12) Patent Application Publication
(10) Pub. No.: US 2017/0319476 A1
(43) Pub. Date: Nov. 9, 2017

(54) Floating Zinc Gastric Delivery Compositions

(71) Applicant: Physician's Seal, LLC, Boca Raton, FL (US)

(72) Inventors: Daniel Hassan, Boca Raton, FL (US); Syed M. Shah, Boca Raton, FL (US); Fred Hassan, Boca Raton, FL (US); Christopher Diorio, Boca Raton, FL (US)

(73) Assignee: Physician's Seal, LLC, Boca Raton, FL (US)

(21) Appl. No.: 15/586,578

(22) Filed: May 4, 2017

Related U.S. Application Data
(60) Provisional application No. 62/332,640, filed on May 6, 2016.
(60) Provisional application No. 62/384,899, filed on Sep. 8, 2016.

(51) Int. Cl. A61K 9/00; A61K 47/02; A61K 33/30; A61K 31/198; A61K 9/20; A61K 47/38; A61K 9/46

(52) U.S. Cl. CPC A61K 9/0065; A61K 47/38; A61K 47/02; A61K 9/0007; A61K 31/198; A61K 9/20; A61K 33/30

(57) ABSTRACT

A composition includes a therapeutically effective oral pharmaceutical dosage form that becomes buoyant upon contact with gastric fluid. The dosage form includes an active ingredient combination including an amino acid source and a zinc source, an anionic polymer, an effervescent agent, and a pH buffer. The dosage form is effective for releasing the active ingredient combination while buoyant on gastric fluid.

### CROSS-REFERENCE TO RELATED APPLICATIONS

This claims priority from U.S. provisional Application No. 62/332,640, filed May 6, 2016, and U.S. provisional Application No. 62/384,899, filed Sep. 8, 2016. Both of these prior applications are incorporated by reference in their entireties.

### FIELD

This relates to the field of therapeutic compositions for gastroesophageal conditions and, more particularly, to floating raft-type active ingredient delivery systems.

### BACKGROUND

Heartburn occurs when the corrosive gastric fluid flows from the stomach into the esophagus through the esophageal sphincter and inflames the lining of the distal esophagus. It is desirable to provide heartburn relief by delivering therapeutically active ingredients to the site of the inflammation, such as the distal esophageal lining.

Floating drug delivery systems are designed to float on top of the gastric fluid in the stomach. They have a bulk density less than the gastric fluid and remain buoyant without substantially affecting the gastric emptying rate. While the delivery system floats, the active ingredients in it gradually releases from the floating matrix.

Floating on top of the gastric fluid improves the residence time of floating drug delivery systems in the zone between the top of the stomach and the distal esophagus. It also focuses delivery of the active ingredients to the site of the disturbance where symptoms originate. This targeted and sustained release of the active ingredients at the site of the disturbance may provide enhanced efficacy for some esophageal conditions compared to other delivery systems.

### BRIEF SUMMARY

A first example of the composition includes a therapeutically effective oral pharmaceutical dosage form that becomes buoyant upon contact with gastric fluid. The dosage form has therein an active ingredient combination including an amino acid source and a zinc source, an anionic polymer, an effervescent agent, and a pH buffer. The dosage form is effective for releasing the active ingredient combination while buoyant on gastric fluid.

A second example of the composition includes a therapeutically effective oral pharmaceutical dosage form that becomes buoyant upon contact with gastric fluid. The dosage form has therein 2% w/w to 10% w/w of an amino acid source; 9% w/w to 45% w/w of a zinc source; 10% w/w to 55% w/w of an anionic polymer; and 1% w/w to 15% w/w of a bicarbonate. The dosage form is effective for releasing the amino acid source and zinc source combination while buoyant on gastric fluid.

An example of a method of treating a gastroesophageal condition associated with stomach acid comprises locally delivering zinc and an amino acid to the distal esophagus of a patient by administering to a patient in need thereof a therapeutically effective oral pharmaceutical dosage form that becomes buoyant upon contact with the patient's gastric fluid. The dosage form has therein an active ingredient combination including an amino acid source and a zinc source, an anionic polymer, an effervescent agent, and a pH buffer. The dosage form releases the active ingredient combination while buoyant on gastric fluid and neutralizes stomach acid while promoting healing of epithelial cells in the distal esophagus.

The following additional features may be included in any of these example compositions and/or methods.

The zinc source may be a water soluble zinc salt.

The anionic polymer may have terminal carboxylate functional groups.

The pH buffer may be capable of maintaining a substantially neutral pH within the dosage form while the dosage form is in contact with stomach acid.

The dosage form may be therapeutically effective for treating a gastroesophageal condition.

The dosage form may be a tablet.

The anionic polymer may swell upon contact with gastric fluid and a ratio of the anionic polymer to the zinc source may be from 1:2 to 2:1.

The anionic polymer may swell upon contact with gastric fluid and the dosage form may include 100 to 500 mg zinc source and 100 to 500 mg of the anionic polymer.

The anionic polymer may swell upon contact with gastric fluid and the dosage form may include 200 to 300 mg zinc source and 200 to 300 mg of the anionic polymer.

The zinc source may be 5% to 50% w/w of the dosage form.

The zinc source may be 20% to 25% w/w of the dosage form.

The anionic polymer may swell upon contact with gastric fluid; the anionic polymer may be 20% to 25% w/w of the dosage form; and the zinc source may be 20% to 25% w/w of the dosage form.

The amino acid source may be 2% to 10% w/w of the dosage form.

### DETAILED DESCRIPTION OF EXAMPLE EMBODIMENTS

The composition may be useful for treating gastroesophageal and gastrointestinal conditions associated with stomach acid contacting the esophageal gut barrier and resulting in gut barrier dysfunction and associated symptoms. Such conditions include, but are not limited to, heartburn, gastritis, GERD, and acid reflux.

The composition is within a dosage form that can be delivered gastrically for sustained delivery of the active ingredients at the site of inflammation. When it reaches the stomach and contacts gastric fluid, it forms a floating raft on top of the gastric fluid, providing a barrier between the stomach acid and upper stomach and esophagus. It then gradually releases the active ingredients from the floating matrix. This is advantageous because the site of irritation for many gastroesophageal and gastrointestinal conditions associated with excess stomach acid is in the interface between the gastric fluid and esophagus, commonly at the distal esophagus.

The damage to the epithelial cells at this interface is mediated through the immune system. Zinc ions attenuate the immune response and may thereby lower the extent of damage and recovery from the immune related cascade that results in gut barrier dysfunction. The composition delivers zinc directly to the site of irritation to treat the underlying condition or its symptoms.

The floating matrix includes an anionic swellable polymer that swells upon contact with gastric acid to form a floating raft atop the gastric acid, which releases the active ingredients therefrom. Examples of such swellable polymers include, but are not limited to, carboxylate or carboxylic acid functionalized swellable polymers such as alginic acid, alginates such as sodium alginate and magnesium alginate, carboxymethyl cellulose (“CMC”) such as sodium CMC, and the like.

One of the active ingredients is zinc from a zinc source. The zinc source may be a pharmaceutically acceptable zinc salt such as, for example, zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate), zinc carnosine, zinc sulfate, and zinc nitrate. In some cases, the zinc source is a water soluble zinc salt.

Anionic polymers, including those with terminal carboxylate groups may form a zinc-polymer complex in which Zn (2+) ions bond to the polymer via its carboxylate ions or other anions. Examples of such zinc-polymer complexes include, but are not limited to, zinc-alginate and zinc-carboxymethyl cellulose. Forming a zinc-polymer complex, may prolong the release of zinc from the floating raft in the stomach.

Alginic acid is a polymer made of mannuronic acid and guluronic acid. The relative concentrations of mannuronic to guluronic acid varies, depending on the source from which the alginate is extracted. A common source of alginic acid is brown algae. Alginate is a salt-form of alginic acid.

CMC is a hydrophilic cellulosic polymer with a molecular weight that varies. Different versions of CMC may have different degrees of carboxylation.

The composition may include an effervescent agent that forms bubbles when it contacts the stomach acid, which helps the polymer matrix remain buoyant atop gastric fluid. Examples of effervescent agents include, but are not limited to, carbonates, bicarbonates, citric acid, and tartaric acid. Carbonates include alkali metal carbonates such as sodium and potassium carbonate and alkali earth metal carbonates such as magnesium and calcium carbonate. Bicarbonates include alkali metal bicarbonates such as sodium and potassium bicarbonate and alkali earth metal bicarbonates such as magnesium and calcium bicarbonate.

The composition may also include a pH buffer. The pH buffer is basic relative to stomach acid and is effective to maintain the internal pH of the floating raft substantially neutral between about 6 and 7.5. Maintaining such a pH within the floating raft helps prevent active ingredients, especially zinc from releasing quickly into the stomach acid. The raft/buffer combination, therefore, effectively insulates the zinc source and the anionic polymer from the stomach's acidic environment having a pH of 1.5 to 3.5. This helps keep the zinc within the floating raft over a longer period of time than would otherwise occur without the pH buffer, providing a sustained release of zinc at the site of inflammation. The amount of pH buffer in the composition is capable of maintaining a substantially neutral pH within the dosage form while the dosage form is in contact with stomach acid.

Examples of pH buffers include, but are not limited to bicarbonate buffers, phosphate buffers, acetic acid, citric acid, and combinations thereof. Bicarbonates such as alkali metal bicarbonates such as sodium and potassium bicarbonate and alkali earth metal bicarbonates such as magnesium and calcium bicarbonate may be particularly useful because they can serve both as an effervescent agent and a pH buffer.

The composition may include an antacid agent. Antacid agents include, but are not limited to, calcium carbonate, sodium bicarbonate, aluminum hydroxide, magnesium hydroxide, a proton pump inhibitor, and/or an H2-blocker.

The composition may include mastic gum or mastic extract. Mastic is a resin from the mastic tree (Pistachia Lentiscus) and has therapeutic properties. According to Publication No. US 2015/0110902, it has been reported to have antibacterial properties and is known to treat gastrointestinal disorders.

The composition may include licorice or licorice extract from the herb Glycyrrhiza glabra. According to reports, licorice is effective in gastric ulcer treatment and has anti-inflammatory effects. Licorice may also raise the concentration of prostaglandins in the digestive system that promote mucus secretion from the stomach. Helicobacter pylori also shows susceptibility to licorice.

It has been reported that zinc intake may reduce the amount of copper in the body. The composition may, therefore, include a source of copper to supplement any loss of copper that the zinc composition may cause. Copper sources may include, but are not limited to, copper gluconate, copper sulfate, copper picolinate, and copper-amino acid complexes.

The composition may include an amino acid source such as a source of individual amino acids and/or oligopeptides. The amino acid source may be useful for helping the body repair epithelial cell damage caused by chronic stomach acid disorders such as chronic heartburn and chronic acid reflux. High protein, polypeptide and amino acids or their combination have been used to restore gut barrier dysfunction (Rao A. & Samak G., “Role of glutamine in protection of intestinal tight junctions,”J Epithel Biol Pharmacol 2012 January; 5(Suppl 1-M7): 47-54).

Examples of amino acid sources include, but are not limited to, sources of glutamine, asparagine, threonine, serine, glycine, arginine, histidine, lysine, aspartic acid, glutamic acid, cysteine, selenocysteine, proline, alanine, valine, isoleucine, leucine, methionine, phenylalanine, tyrosine, and tryptophan. Any of these amino acids, combinations thereof, and/or oligopeptides thereof that are therapeutically effective for treating cell damage caused by stomach acid may be used. When included in the composition, the amino acid source gradually releases from the floating raft.

Glutamine, by way of example, has been reported to reduce bacterial translocation following abdominal exposure to radiation in rats by maintaining the gut's mucosal barrier. See, Souba et al., Journal of Surgical Research, Vol. 48, pgs 1-5 (1990). Although not intending to be bound by theory, amino acid-containing ingredients such as glutamine may help maintain the stomach's mucosal barrier as well. The co-administration of the amino acid-containing ingredients along with the zinc may have a combined therapeutic effect as reported by Wapnir, R. A., Zinc Deficiency, Malnutrition and the Gastrointestinal Tract, J. Nutr. 130: 1388S-1392S, 2000.

The composition may include melatonin, which has been suggested to be involved with improving GERD symptoms as reported by Patrick in Alternative Medicine Review, Vol. 16 No. 2, pages 116-133 (2011).

The composition may include at least one H2 blocker, which is an active ingredient that reduces the amount of acid made by the stomach. H2 blockers are also called histamine H2-receptor antagonists. Examples of H2 blockers include cimetidine, famotidine, nizatidine, and ranitidine.

The dosage form is administered in a therapeutically effective amount. A therapeutically effective amount is an amount effective to achieve a desired therapeutic benefit, such as an amount effective to prevent, alleviate, ameliorate, or treat the underlying causes and/or symptoms of the physiological condition being treated.

For some uses of the composition, a therapeutically effective amount is an amount effective to decrease stomach acid. For some uses of the composition, a therapeutically effective amount may be an amount effective to reduce the patient's symptoms caused by the condition being treated.

When the composition is administered to treat a condition associated with heartburn, for example, a therapeutically effective amount may be an amount effective to reduce the clinical symptoms of the condition, such as by reducing the patient's reports of experiencing heartburn. The patient may be a human or animal.

In humans, a therapeutically effective amount range for an active ingredient is often 1-2,000 mg/day, including 1-25 mg/day, 25-50 mg/day, 50-75 mg/day, 75-100 mg/day, 100-150 mg/day, 150-200 mg/day, 200-250 mg/day, 250-300 mg/day, 300-350 mg/day, 350-400 mg/day, 400-450 mg/day, 450-500 mg/day, 500-550 mg/day, 550-600 mg/day, 600-650 mg/day, 650-700 mg/day, 700-750 mg/day, 750-800 mg/day, 800-850 mg/day, 850-900 mg/day, 900-950 mg/day, 950-1,000 mg/day. Higher doses (1,000-3,000 mg/day) might also be effective. The weight in mg is often calibrated to the body weight of the subject in kg, thus these example doses may also be written in terms of mg/kg of body weight per day.

In practice, the therapeutically effective amount may vary depending on numerous factors associated with the patient, including age, weight, height, severity of the disorder, administration technique, and other factors. The therapeutically effective amount may be determined by medical personnel taking into account the relevant circumstances.

The dosage form may be administered as a single dose or as part of a dosage regimen. For a dosage regimen, the therapeutically effective amount is adjustable dose to dose to provide a desired therapeutic response.

Multiple doses may be administered at a predetermined time interval and subsequent doses may be proportionally reduced, depending on the situation. By administering the dosage form as part of a dosage regimen, local concentrations may be allowed to reach a desired concentration of the zinc composition.

Table 1 is a list of a few of the possible therapeutically effective amounts of active ingredients in examples of the composition. Any combination of the amounts in Table 1 may be used in combination with each other. It is not necessary for every example of the composition to include all four active ingredients.

TABLE 1
Examples of therapeutically effective amounts

| Active | Mass (milligrams) | | | | |
|---|---|---|---|---|---|
| Zinc source | 100-500 | 150-400 | 150-300 | 225-275 | 175-225 |
| Amino acid | 25-100 | 30-90 | 40-60 | 45-55 | 46-54 |
| source | | | | | |
| Licorice | 100-400 | 150-300 | 150-250 | 175-225 | 190-210 |
| extract | | | | | |
| mastic | 100-400 | 150-300 | 150-250 | 175-225 | 190-210 |

The dosage form may be administered via a number of techniques. The administration techniques will involve providing the dosage form to the stomach such as by oral or other gastric administration technique.

The composition may be administered in a pharmaceutically acceptable dosage form such as a powders, granule, tablet, pill, capsule, suppository, and sachet. They also include liquid dosage forms, such as dispersions, syrups, suspensions, emulsions, and other solutions. In such a case, the composition and other ingredients may be blended with one or more pharmaceutically acceptable excipients.

Exemplary excipients include, but are not limited to, carriers, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, emollients, dissolution enhancers, dispersing agents, or combinations thereof.

Tablets and caplets may be prepared using conventional tableting techniques such as dry blending or wet granulation. The dry blend or granulation may be compressed into a final tablet form.

Capsules may be prepared using different techniques. For example, dried granules produced by wet granulating the ingredients may be loaded into a capsule, such as a gelatin capsule.

Conventional processing aids may be used to prepare dosage form. Examples of processing aids include, but are not limited to, magnesium stearate, stearic acid, talc, and sodium lauryl sulfate.

The dosage form may include a pharmaceutically acceptable filler. Examples of fillers include, but are not limited to, microcrystalline cellulose, silicates, calcium carbonate, glycine, dextrin, sucrose, sorbitol, dicalcium phosphate, calcium sulfate, lactose, kaolin, mannitol, sodium chloride, talc, dry starches and powdered sugar.

The dosage form may include a pharmaceutically acceptable binder. Examples of binders include, but are not limited to, sugars such as sucrose and glucose; natural binders such as starch, cellulose, and gelatin; and polymers such as methyl cellulose, ethyl cellulose, hydroxypropylmethyl cellulose, hydroxypropyl cellulose, sodium carboxymethyl cellulose, microcrystalline cellulose, silicified microcrystalline cellulose, polyvinyl pyrrolidone, polyethylene glycol, polyvinyl alcohols, and polymethacrylates.

The dosage form may be coated to aid in swallowing, to mask the taste of the ingredients, improve appearance, to protect the dosage form from moisture, and/or to have an enteric coating. The coating may be applied using conventional coating techniques, such as, for example, spray coating, bed coating, or the like.

Several particular examples of the dosage form will now be described. The scope of possible embodiments, however, is not limited these examples. These examples are presented by % w/w of the dosage form. Any combination of the % w/w listed below may be employed

The anionic polymer may be 10% w/w to 55% w/w, 10% w/w to 40% w/w, 20% w/w to 30% w/w, or 20%w/w to 25% w/w of the dosage form.

The zinc source may be 5% w/w to 50% w/w, 9% w/w to 45% w/w, 20% w/w to 30% w/w, or 20% to 25% w/w of the dosage form.

The amino acid source may be 2% w/w to 10% w/w, 3% w/w to 8% w/w , or 4% w/w to 6% w/w of the dosage form.

Licorice extract may be 8% w/w to 35% w/w, 10% w/w to 25% w/w, 10% w/w to 20% w/w of the dosage form.

Mastic may be 8% w/w to 35% w/w, 10% w/w to 25% w/w, 10% w/w to 20% w/w of the dosage form.

A bicarbonate may be 1% w/w to 15% w/w, 1% w/w to 15%, w/w, 5% w/w to 15% w/w, or 7% w/w to 12% w/w of the dosage form.

The amount of melatonin may be, for example, 0.1% w/w to 60% w/w, 0.1% w/w to 40% w/w, or 0.1% w/w to 25% w/w, or 0.1% w/w to 10% w/w of the dosage form.

The amount of antacid in the composition may be, for example, 0.1% to 60% w/w, 0.1% to 40% w/w, or 0.1% to 25% w/w, or 0.1% to 10% w/w of the composition.

In some examples of the dosage form, the ratio of the anionic polymer to the zinc source is from 1:2 to 2:1.

### EXAMPLES

This section describes a few specific examples of the composition. These examples are presented by way of example only and are not intended to limit the scope of the possible embodiments.

Tables 2 and 3 list ingredients of examples of a tablet dosage form, including suitable ranges for the amount of each ingredient.

TABLE 2
Contents of an example dosage form

| | Ingredient | mg | range (mg) |
|---|---|---|---|
| Intragranular | Zinc Chloride | 250 | 100-500 |
| | sodium alginate | 250 | 100-500 |
| | sodium carboxy | 75 | 50-150 |
| | methylcellulose or carbopol | | |
| | silicified microcrystalline | 150 | 10-200 |
| | cellulose | | |
| Extragranular | silicified microcrystalline | 200 | 10-300 |
| | cellulose | | |
| | sodium carboxy | 75 | 50-150 |
| | methylcellulose or carbopol | | |
| | magnesium stearate | 7.5 | 1-15 |
| | sodium bicarbonate | 100 | 15-150 |
| | Total tablet weight | 1107.5 | |

TABLE 3
Contents of an example dosage form

| | Ingredient | mg | range (mg) |
|---|---|---|---|
| Intragranular | Zinc Chloride | 200 | 100-500 |
| | sodium alginate | 200 | 100-500 |
| | glutamine | 50 | 25-100 |
| | sodium carboxy | 75 | 50-150 |
| | methylcellulose | | |
| | silicified microcrystalline | 100 | 10-200 |
| | cellulose | | |
| Extragranular | silicified microcrystalline | 150 | 10-300 |
| | cellulose | | |
| | licorice extract | 200 | 100-400 |
| | sodium carboxy | 75 | 100-300 |
| | methylcellulose | | |
| | magnesium stearate | 7.5 | 1-15 |
| | sodium bicarbonate | 100 | 15-150 |
| | Total tablet weight | 1157.5 | |

In these examples, the zinc source is zinc chloride and the anionic polymer is sodium alginate. The example in Table 3 includes glutamine as the amino acid source and licorice extract as an additional active ingredient.

Different processes may be used to prepare tablets of these examples.

Wet granulation. To prepare the internal granulation, the zinc chloride, sodium alginate, sodium phosphate dibasic, sodium CMC and/or carbopol and silicified MCC are added to a high shear mixer and dry blended until sufficiently mixed. The resulting blend is then wet granulated with water until a suitable granulation is formed. The wet mass is then dried in a fluid bed dryer or in an oven on trays until a moisture level of 4-9% is obtained. The dried granules are then screen or milled with a cone mill to achieve a uniform particle size.

To prepare the external granulation, silicified MCC, sodium CMC and/or carbopol, and sodium bicarbonate is dry mixed in a V blender or equivalent for 10 minutes or until uniform.

The internal granulation is then added to the V blender and mixed until a uniform blend is achieved. Magnesium stearate is then added and blended for 5 minutes. The final blend can then be compressed into tablet on a rotary tablet press. A colored film coat of hypromellose or polyvinyl alcohol with color can then be applied in a standard pan coater for appearance and to improve swallowability.

Dry granulation. Alternatively, the tablets are prepared via a dry granulation or roller compaction process.

To prepare the internal granulation, the zinc chloride, sodium alginate, sodium phosphate dibasic, sodium CMC and/or carbopol and silicified MCC are added to a V blender and dry blended until sufficiently mixed. The resulting blend is then passed through a roller compactor until suitable compacts are formed, magnesium stearate can be used to reduce sticking during the compaction process. The compacts are then screen or milled with a cone mill to achieve a uniform particle size.

To prepare the external granulation, silicified MCC, sodium CMC and/or carbopol, and sodium bicarbonate is dry mixed in a V blender or equivalent for about 10 minutes or until uniform.

The sized internal granulation is then added to the V blender and mixed until a uniform blend is achieved. Magnesium stearate is then added and blended for 5 minutes. The final blend can then be compressed into tablet on a rotary tablet press. A colored film coat of hypromellose or polyvinyl alcohol with color can then be applied in a standard pan coater for appearance and to improve swallowability.

Direct blending. Alternatively, the tablets are prepared via a direct blending process.

The zinc chloride, sodium alginate, sodium phosphate dibasic, sodium CMC and/or carbopol, silicified MCC, and sodium bicarbonate are dry mixed in a V blender or equivalent for 10-20 minutes or until uniform. Magnesium stearate is then added and blended for about 5 minutes. The final blend is then compressed into tablet on a rotary tablet press. A colored film coat of hypromellose or polyvinyl alcohol with color can then be applied in a standard pan coater for appearance and to improve swallowability. The final blend can also be filled into hard shell two piece capsules on any suitable capsule filler.

This disclosure has described example embodiments but not all possible embodiments of the composition or methods. Where a particular feature is disclosed in the context of a particular embodiment, that feature can also be used, to the extent possible, in combination with and/or in the context of other embodiments. The composition and related methods may be embodied in many different forms and should not be construed as being limited to only the embodiments described here.

That which is claimed is:

1. A composition comprising:
a therapeutically effective oral pharmaceutical dosage form that becomes buoyant upon contact with gastric fluid, the dosage form having therein:
an active ingredient combination including an amino acid source and a zinc source;
an anionic polymer;
an effervescent agent; and
a pH buffer;
wherein the dosage form is effective for releasing the active ingredient combination while buoyant on gastric fluid.

2. The composition of claim 1, wherein the zinc source is a water soluble zinc salt.

3. The composition of claim 1, wherein the anionic polymer has terminal carboxylate functional groups.

4. The composition of claim 1, wherein the pH buffer is in an amount capable of maintaining a substantially neutral pH within the dosage form while the dosage form is in contact with stomach acid.

5. The composition of claim 1, wherein the dosage form is therapeutically effective for treating a gastroesophageal condition.

6. The composition of claim 1, wherein the dosage form is a tablet.

7. The composition of claim 1, wherein the anionic polymer will swell upon contact with gastric fluid and a ratio of the anionic polymer to the zinc source is from 1:2 to 2:1.

8. The composition of claim 1, wherein the anionic polymer will swell upon contact with gastric fluid and the dosage form includes 100 to 500 mg zinc source and 100 to 500 mg of the anionic polymer.

9. The composition of claim 1, wherein the anionic polymer will swell upon contact with gastric fluid and the dosage form includes 200 to 300 mg zinc source and 200 to 300 mg of the anionic polymer.

10. The composition of claim 1, wherein the zinc source is 5% to 50% w/w of the dosage form.

11. The composition of claim 1, wherein the zinc source is 20% to 25% w/w of the dosage form.

12. The composition of claim 1, wherein the anionic polymer will swell upon contact with gastric fluid;
the anionic polymer is 20% to 25% w/w of the dosage form; and
the zinc source is 20% to 25% w/w of the dosage form.

13. The composition of claim 1, wherein the amino acid source is 2% to 10% w/w of the dosage form.

14. A composition comprising:
a therapeutically effective oral pharmaceutical dosage form that becomes buoyant upon contact with gastric fluid, the dosage form having therein:
2% w/w to 10% w/w of an amino acid source;
9% w/w to 45% w/w of a zinc source;
10% w/w to 55% w/w of an anionic polymer;
1% w/w to 15% w/w of a bicarbonate; and
wherein the dosage form is effective for releasing the amino acid source and zinc from the zinc source while buoyant on gastric fluid.

15. The composition of claim 14, wherein the zinc source is a water soluble zinc salt.

16. The composition of claim 14, wherein the anionic polymer has terminal carboxylate functional groups.

17. The composition of claim 14, wherein an amount of the bicarbonate is capable of maintaining a substantially neutral pH within the dosage form while the dosage form is in contact with stomach acid.

18. The composition of claim 14, wherein the dosage form is therapeutically effective for treating a gastroesophageal condition.

19. The composition of claim 14, wherein the dosage form is a tablet.

20. The composition of claim 14, wherein the anionic polymer will swell upon contact with gastric fluid and a ratio of the anionic polymer to the zinc source is from 1:2 to 2:1.

21. The composition of claim 14, wherein:
the anionic polymer will swell upon contact with gastric fluid;
the anionic polymer is 20% to 25% w/w of the dosage form; and
the zinc source is 20% to 25% w/w of the dosage form.

22. The composition of claim 14, wherein the anionic polymer will swell upon contact with gastric fluid and the dosage form includes 200 to 300 mg zinc source and 200 to 300 mg of the anionic polymer.

23. The composition of claim 14, wherein the amino acid source includes glutamine.

24. A method of treating a gastroesophageal condition associated with stomach acid, the method comprising locally delivering zinc and an amino acid to a distal esophagus of a patient by administering to a patient in need thereof:
a therapeutically effective oral pharmaceutical dosage form that becomes buoyant upon contact with the patient's gastric fluid, the dosage form having therein:
an active ingredient combination including an amino acid source and a zinc source;
an anionic polymer;
an effervescent agent; and
a pH buffer;
wherein the dosage form releases the active ingredient combination while buoyant on gastric fluid and neutralizes stomach acid while promoting healing of epithelial cells in the distal esophagus.

25. The method of claim 24, wherein the zinc source is a water soluble zinc salt.

26. The method of claim 24, wherein the anionic polymer has terminal carboxylate functional groups.

27. The method of claim 24, wherein the pH buffer is capable of maintaining a substantially neutral pH within the dosage form while the dosage form is in contact with stomach acid.

28. The method of claim 24, wherein the dosage form is therapeutically effective for treating a gastroesophageal condition.

29. The method of claim 24, wherein the dosage form is a tablet.

30. The method of claim 24, wherein the anionic polymer will swell upon contact with gastric fluid and a ratio of the anionic polymer to the zinc source is from 1:2 to 2:1.

31. The method of claim 24, wherein the anionic polymer will swell upon contact with gastric fluid and the dosage form includes 100 to 500 mg zinc source and 100 to 500 mg of the anionic polymer.

32. The method of claim 24, wherein the anionic polymer will swell upon contact with gastric fluid and the dosage form includes 200 to 300 mg zinc source and 200 to 300 mg of the anionic polymer.

33. The method of claim 24, wherein the zinc source is 5% to 50% w/w of the dosage form.

34. The method of claim 24, wherein the zinc source is 20% to 25% w/w of the dosage form.

35. The method of claim 24, wherein the anionic polymer will swell upon contact with gastric fluid;
the anionic polymer is 20% to 25% w/w of the dosage form; and
the zinc source is 20% to 25% w/w of the dosage form.

36. The method of claim 24, further comprising 2% to 10% w/w of an amino acid source.
